(19) United States
(12) Patent Application Publication
(10) Pub. No.: US 2024/0288578 A1
(43) Pub. Date: Aug. 29, 2024

(54) METHOD AND SYSTEM FOR CONTROLLING A WAVE-TRANSMITTING DEVICE

(71) Applicant: SUPERSONIC IMAGINE, Aix-En-Provence (FR)

(72) Inventors: Hubert CORDONNIER, Aix en Provence (FR); Jean-Alain MARTIN, Aix en Provence (FR); Guillaume ROUX, Aix-en-Provence (FR)

(21) Appl. No.: 18/424,574

(22) Filed: Jan. 26, 2024

(30) Foreign Application Priority Data
Feb. 27, 2023 (FR) 2301783

(51) Int. Cl. G01S 15/89; G01S 7/52

(52) U.S. Cl. CPC G01S 15/8909; G01S 7/5202

(57) ABSTRACT

Examples of the disclosure relate to a method and a system for controlling a transmitting device including a transducer for transmitting waves, the method including a modulation of output signals emitted from a pulser in order to control the transducer, the modulation being performed by a controller according to at least one of control of a supply voltage of the pulser by a supply controller connected to a supply terminal of the pulser, and control of return signals from the transducer by a return controller connected to a return terminal of the transducer.

### BACKGROUND

A wave-transmitting device usually comprises a plurality of transducer elements (for example disposed in an array) for communication, imaging or scanning purposes, for example in the field of imaging, in particular medical imaging, radar, sonar, seismology, wireless communications, radio astronomy, acoustics and biomedicine. One example comprises ultrasound imaging.

For this purpose, the wave-transmitting device can be controlled by means of electrical signals emitted by one or more pulsers. These signals define waves transmitted to transducer elements of the transmitting device, causing waves to be emitted into a given medium. Electrical signals can be picked up in return by the transducer elements, these same transducers or other transducers, these signals representing a response (or echo) of the medium to wave excitation.

For example, the aim of the ultrasound imaging may be to estimate the reflectivity of a medium. The frequency of the signals may therefore be selected depending on the characteristics of the observed medium (for example human tissue).

In particular, in a conventional method for ultrasonic imaging, it is possible to use, for example, an ultrasonic transducer device equipped with at least one ultrasonic transducer element to convert electrical signals into ultrasound waves. Each transducer can thus transmit one or successively more ultrasound beams towards a medium, which corresponds to a transmission operation. Subsequently, in a receiving operation, a set of backscattered echo signals can be received from the medium by the same set or another set of transducer elements. In particular, each of the transducer elements can, for example, convert a received echo signal into an electrical signal. The signal can subsequently be processed by the ultrasound system or by any associated (dedicated) system, which is directly connected or not. For example, the signal can be amplified, filtered, digitised and/or a signal conditioning operation may be carried out. The transducer elements may be disposed as a line of transducers, a matrix, or as an array of transducers or any other configuration.

However, it has become clear that certain wave-emitting devices, in particular imaging systems (ultrasound or other types of imaging), suffer from a problem of accelerated ageing, which can result in the occurrence of faults, deterioration of the system and/or poor system performance. These issues of faults or performance degradation can lead to various failures, in particular poor image quality in the case of imaging applications (for example ultrasound imaging).

Moreover, the development of such wave-emitting devices is often complex, which can make the design and/or calibration of such devices particularly complicated and costly.

### DESCRIPTION OF THE DISCLOSURE

One of the objects of the present invention is to resolve at least one of the abovementioned problems or shortcomings.

In particular, it may be desirable to improve the reliability and/or performance of a wave-emitting device, in particular, but not exclusively, of an ultrasonic transducer device.

In particular, it may be desirable to improve the control of a wave-emitting device, in particular, but not exclusively, of an ultrasonic transducer device.

To this end, according to a first aspect, the present invention relates to a method for controlling a wave-transmitting device comprising a transducer for transmitting waves. The method according to the first aspect comprises:
modulation of output signals emitted from a pulser in order to control the transducer, said modulation being performed by a controller according to at least one of:
a) control of a supply voltage of the pulser by a supply controller connected to a supply terminal of the pulser; and
c) control of return signals from the transducer by a return controller connected to a return terminal of the transducer.

The provision of such a method makes it advantageously possible to improve the control of the wave-transmitting device and thus the performance and reliability of the system overall. The modulation makes it possible in particular to better control the output signals sent to the transducer and thus to ensure that the theoretical limit values of the parameters (for example, in current, voltage and/or power) of the transducer, and more generally of the transmission chain from the pulser to the transducer, are respected. By controlling the transducer more precisely, it is possible to avoid over-stressing the system, limit failures (faults, degradation, etc.), ensure a good level of reliability of the system and guarantee that the system has satisfactory performance.

Thanks to the concept of the present invention, it is possible to adapt the waves sent with more flexibility (in power, frequency, duration, etc.) without impairing the pulsers or transducers, which makes it possible to overcome certain compromises or limitations (particularly in terms of power) usually associated with this type of device to guarantee a good performance and normal ageing of the whole unit. In contrast to conventional systems, the concept of the present invention makes it possible to configure the device so as to transmit the desired waves, i.e. in accordance with the desired wave characteristics for the intended application.

With limited modification to the whole unit, and thus a small additional cost, it is thus possible to obtain a wave-emitting device with high-level capabilities.

It is possible, for example, to improve the image quality, such as the quality of an ultrasound image, in an ultrasound imaging application by implementing the concept of the present invention. Thanks to the method of the present invention, it is in particular possible to obtain a good image quality.

The method according to the invention may comprise other features, which may be adopted separately or in combination, in particular among the following embodiments, which are presented for illustrative purposes only and may be combined or associated unless otherwise specified.

According to one example, during the control a), the supply controller modulates a power supplied to the pulser on the supply terminal from a static voltage supplied by a voltage regulator.

According to one example, the supply terminal of the pulser to which the supply controller is connected is one of: a positive supply terminal, a negative supply terminal and a ground terminal of the pulser.

According to one example, the modulation is carried out by means of one (or at least one) said supply controller, the modulation being configured to cause a variation over time of the supply voltage to the pulser according to at least two distinct non-zero values.

According to one example, during the control b), the output controller modulates at least one of the electrical characteristics of the output signals of the pulser out of voltage, current and power of said output signals.

According to one example, the return terminal of the transducer is connected in series to a ground of the transmitting device.

According to one example, the output signals are modulated at a modulation frequency Fm greater than or equal to a fraction 1/P of the pulse frequency Ft at which the transducer transmits waves in response to the output signals from the pulser, P being an integer equal to 10.

According to one example, the transmitting device comprises a plurality of transducers each controlled by a respective pulser, signal modulation being performed independently per channel between each transducer and the respective pulser.

According to one example, the method comprises:
measuring at least one operating parameter of a transmission chain via which the pulser sends the output signals to the transducer;
wherein the output signals are modulated as a function of said at least one measured operating parameter.

According to one example, said at least one operating parameter comprises at least one of:
a voltage at the pulser output;
the supply voltage of the pulser;
a ground voltage of the pulser;
a voltage at the terminals of the transmitting device;
a voltage received from the transducer;
a current at the pulser output;
a supply current of the pulser;
a ground current of the pulser;
a current flowing through the transmitting device;
a temperature of the pulser;
a temperature of the transmitting device;
an electric field emitted by the pulser;
an electric field emitted by the transmitting device;
a magnetic field emitted by the pulser;
a magnetic field emitted by the transmitting device;
a sound pressure emitted by the transmitting device; and
a sound pressure received by the transmitting device.

According to one example, the method comprises:
comparing said at least one operating parameter with respectively one threshold value;
the modulation of the output signals being a function of a result of said comparison.

According to one example, the supply controller regulates the supply voltage to a first value during the control a) if said at least one operating parameter does not exceed a respective threshold value, and
wherein the control a) comprises adapting the supply voltage from the first value to a second value that is different from the first value if said at least one operating parameter exceeds said respective threshold value.

According to one example, the output signals are modulated by slaving at least one of the controls a), b) and c) as a function of said at least one measured operating parameter.

According to one example, said at least one operating parameter is measured during a calibration of the transmission chain, wherein the modulation is carried out after the calibration as a response to at least one command determined as a function of said at least one operating parameter.

According to one example, the method comprises:
measuring at least one operating parameter of transmission chains via which each transducer receives output signals from a respective pulser; and
upon detection that an operating parameter of a transmission chain meets a predetermined criterion, adapting the modulation of the output signals sent to the transducer of said transmission chain.

According to one example, the output signals are modulated as a function of at least one of:
an imaging mode implemented by the pulser to control the transmitting device;
a type of transmitting device; and
user parameters of the transmitting device.

According to one example, the transducer is a piezoelectric transducer.

According to one example, said method is applied to medical ultrasound imaging.

According to a second aspect, the present invention may involve a computer program comprising instructions, which, when the program is run by a computer or more precisely by a control system driving a transducer device, contributes to the implementation of the method according to the first aspect. In particular, the various steps of the method according to the first aspect may involve instructions from computer programs.

Such a computer program can use any programming language or equivalent, and it may be in the form of source code, object code, or intermediate code between a source code and an object code, such as in a partially compiled form, or in any other desirable form.

According to a third aspect, the present invention relates to a recording medium (or storage medium), readable by a computer (or processor), on which a computer program according to the third aspect of the present invention is stored.

On the one hand, the recording medium may be any entity or device capable of storing the program, such as at least one volatile and/or non-volatile memory. For example, the medium may comprise a storage means, such as a rewritable non-volatile memory, a ROM memory, a CD-ROM or a ROM memory of the microelectronic circuit type, or a magnetic recording means or a hard disk. This memory can, for example, comprise a graphics card (or video card) memory, this type of memory being particularly suitable for processing image data (or video data).

On the other hand, this recording medium may also be a transmissible medium such as an electrical or optical signal, such a signal being able to be conveyed via an electrical or optical cable, by conventional or Hertzian radio or by self-directed laser beam or by other means. The computer program according to the present invention can be downloaded via a wired or wireless network, local or otherwise (e.g. Bluetooth®, Wi-Fi, Ethernet, Internet, 4G, 5G or others).

Alternatively, the recording medium may be an integrated circuit in which the computer program is incorporated, the integrated circuit being adapted to execute or to be used in the execution of the method in question.

According to a fourth aspect, the present invention relates to a control system configured to control a wave-transmitting device comprising a transducer for emitting waves, and this by implementing the control method of the first aspect of the present invention.

According to one example, the control system comprises a memory associated with a processor, this memory comprising a computer program according to the second aspect of the invention.

According to one example, the control system comprises:
a pulser; and
a controller configured to modulate output signals transmitted from the pulser to control the transmitting device, said controller comprising at least one of:
a) a supply controller, connected to a supply terminal of the pulser, configured to control a supply voltage of the pulser; and
c) a return controller, connected to a return terminal of the transducer, configured to control return signals from the transducer.

The control system may have functionalities that correspond to the operations of the method according to the first aspect of the invention. In particular, the different embodiments mentioned above in relation to the method according to the first aspect of the invention as well as the associated advantages can be applied in a similar way to the control system according to the fourth aspect of the present invention.

The features and advantages of the invention will become clearer upon reading the following description, given only by way of non-limiting example, and made with reference to the appended figures. In particular, the examples illustrated in the figures may be combined unless there is significant inconsistency.

### BRIEF DESCRIPTION OF THE FIGURES

Other features and advantages of the present invention will become apparent from the description of the non-limiting exemplary embodiments of the present invention below, with reference to the appended FIGS. 1 to 15, wherein:

FIG. 1 shows a schematic drawing of a system for controlling a wave-transmitting device according to exemplary embodiments of the present invention;

FIG. 2 shows a schematic drawing of a system for controlling a wave-transmitting device according to exemplary embodiments of the present invention;

FIG. 3 shows a schematic drawing of an ultrasound imaging system according to an exemplary embodiment of the present invention;

FIG. 4 shows a schematic drawing of a system for controlling an ultrasonic transducer device according to exemplary embodiments of the present invention;

FIG. 5 schematically shows a controller configured to modulate the output signals from a pulser of the system of FIG. 4 according to exemplary embodiments of the present invention;

FIG. 6 is a diagram schematically showing a control method using the control system in FIG. 4 according to exemplary embodiments of the present invention;

FIG. 7 schematically shows the modulation of output signals from a pulser of the control system of FIG. 4 according to exemplary embodiments of the present invention;

FIG. 8 schematically shows the modulation of output signals from a pulser of the control system of FIG. 4 according to exemplary embodiments of the present invention;

FIG. 9 schematically shows the modulation of output signals from a plurality of a pulsers of the control system of FIG. 4 according to exemplary embodiments of the present invention;

FIG. 10 shows a schematic drawing of a system for controlling an ultrasonic transducer device according to exemplary embodiments of the present invention;

FIG. 11 is a diagram schematically showing a control method using the control system in FIG. 10 according to exemplary embodiments of the present invention;

FIG. 12 schematically shows means for measuring a current in the control system of FIG. 10 according to exemplary embodiments of the present invention;

FIG. 13 schematically shows means for measuring a current in the control system of FIG. 10 according to exemplary embodiments of the present invention;

FIG. 14 schematically shows means for measuring a voltage in the control system of FIG. 10 according to exemplary embodiments of the present invention; and

FIG. 15 schematically shows means for measuring power in the control system of FIG. 10 according to exemplary embodiments of the present invention.

### DESCRIPTION OF THE EMBODIMENTS

The present invention relates to systems for controlling a wave-transmitting device and methods for controlling such a transmitting device. This transmitting device may be an ultrasound device, although other types are possible. In particular, it may be an imaging device, for example a medical imaging device, such as a medical ultrasound device (or system).

A wave-transmitting device, such as a transducer device for example, can be controlled by means of electrical signals emitted by one or more electronic pulsers, known more simply as “pulsers”, i.e. generators of electrical signals. Each transducer (for example ultrasonic transducer) of the transmitting device can thus be controlled by the signals supplied by a pulser of a control system. These electrical signals can be transmitted by a transmission chain from the pulsers to the transducers of the transmitting device.

However, it has been observed that the control of such a transmitting device, for example in an imaging application (using ultrasound for example), is not always properly mastered. Indeed, the signals emitted by the pulser(s) do not always respect the limits established for each component of the system. It has been observed that the limit values set for certain parameters (current, voltage, temperature, etc.) of components in the chain(s) of transmission of the system can be exceeded or not complied with, which can lead to a deterioration or decline in performance of the system and/or to premature ageing thereof.

These constraints and/or problems of performance and reliability can have multiple origins. Firstly, the pulsers are components configured to generate signals of relatively high voltage (for example of the order of ±100V) and relatively high current (for example of the order of ±2A). In certain cases, it has been observed that a pulser can generate output signals with too high a voltage and/or current, which can impair the system (for example cause a rise in temperature and a malfunction of the pulser) or cause premature ageing of the system. Other elements of the system can also deteriorate as a result of the stress caused by these excess voltages and/or currents.

In addition, in certain ultrasound imaging systems, the piezoelectric transducers of the transmitting device are controlled in transmission by pulsers generating signals that may contain harmonics, in particular when these signals are square-shaped. However, these harmonics are not generally converted into acoustic energy (or only to a limited extent) by the piezoelectric transducers and cause heat dissipation, which limits the energy efficiency of the system insofar as certain parts of the signals emitted by the pulsers place a strain on the system without being useful for controlling the transducer. Thus, the unused energy passing through a pulser can reduce the thermal and electric performance of the pulser. The unused energy radiated by the system can also reduce the electromagnetic performance of the system. Finally, the unused energy converted into heat by the transducer can reduce the thermal performance of the transducer.

Moreover, part of the harmonics can be converted by the transducer and end up in the waves transmitted to the external environment. In certain applications, the presence of such harmonics is not desirable, including in the acoustic field. The emission of these harmonics may mean that the acoustic signals emitted by the system are different from the theoretical target signals, which may lead in particular to a poorer image quality in the case of an imaging application (ultrasound imaging for example).

In certain cases, the emission of such harmonics leads to excessive electromagnetic radiation, which can be problematic in particular when a maximum limit of this radiation has to be adhered to (for example in compliance with a regulation imposed to obtain a certification of the system).

In order to limit the harmonics and prevent excessive electromagnetic radiation, it is known to use linear pulsers able to generate sinusoidal signals, but this technique is costly and is not very effective, particularly in the medical field, in particular because of the fact that the waves generated are limited in power and produce a lot of heat.

The abovementioned constraints and/or problems therefore make it necessary to make compromises or limit certain characteristics of such a wave-transmitting device in order to guarantee good performance and acceptable ageing, or even optimum ageing according to market requirements, of the whole unit. These constraints and/or problems can also make it difficult to qualify such a wave-transmitting device according to current standards.

The present invention advantageously makes it possible in particular to solve the abovementioned problems and shortcomings thanks to the introduction of one or more controllers in a system for controlling a wave-transmitting device, this or these controller(s) making it possible to modulate the output signals of one or more pulsers in a dynamic way, for example in real time (or near real time). In contrast to conventional control systems, the present invention allows precise and flexible control over time of the signals emitted at the output of a pulser to a transducer of the transmitting device.

Control methods and control systems for controlling a wave-transmitting device will be described below according to particular embodiments of the invention with joint reference to FIGS. 1-15. Unless otherwise stated, elements common to or similar to several figures bear the same reference numbers and have identical or similar features such that these common elements are not generally described again for the sake of brevity.

The terms “first” (or first, “second”, etc.) are used in this document by arbitrary convention to identify and distinguish between different elements (such as operations, devices, etc.) implemented in the embodiments described hereinafter.

As previously stated, the present invention relates in particular to a method for controlling a wave-transmitting device. FIG. 1 is a schematic representation of a system (or device) 10, also referred to as a control system, configured to control a wave-transmitting device 20 according to exemplary embodiments of the present invention.

The device 20 is configured to transmit, and possibly also receive, waves W. The nature of these waves W depends on the nature and the configuration of the device 20, particularly in view of the use made of it. According to one example, the device 20 is configured to transmit waves W1. According to another example, the device 20 is configured to transmit waves W1 and receive waves W2.

The waves W can, for example, be (or comprise) sound waves, for example of the ultrasound type. By way of example, the device 20 will hereinafter be considered to be an ultrasonic transducer device (or ultrasound probe) configured to transmit ultrasound waves W1. It should be noted, however, that other examples of a wave-transmitting device are possible according to the present invention as stated earlier. In particular, all or part of the wave-transmitting device of the present invention can be deployed in a probe, for example an ultrasound probe. In other words, all or part of the device of the invention can be external to the probe.

In this example, the control system 10 can comprise a processing unit (or module) 11 and a control unit (or module) 12.

The control unit 12 (also referred to as power control unit) is configured to control the transducer device 20 by means of electrical signals SG1, which are exchanged (or transmitted) between the system 10 and the transducer device 20. The control unit 12 thus generates electrical signals SG1, which are transmitted to the transducer device 20 to cause the emission, by said transducer device 20, of ultrasound waves W1 towards and/or into a medium M. The electrical signals SG1 thus generated are representative of (or define) the ultrasound waves W1 projected into the medium M. To this end, the control unit 12 can, for example, be or comprise at least one electronic pulser, also referred to as “pulser”. The electrical signals SG1 can be of various shapes, for example square, sinusoidal, random (or pseudo-random), any shape, etc.

The control unit 12 can optionally comprise receiver devices or receiver circuits (not shown) configured to receive electrical signals SG1 coming from the transducer device 20.

The processing unit 11 can be configured to control the control unit 12, for example by controlling the electrical signals SG1. This processing unit 11 can, for example, be or comprise at least one processor.

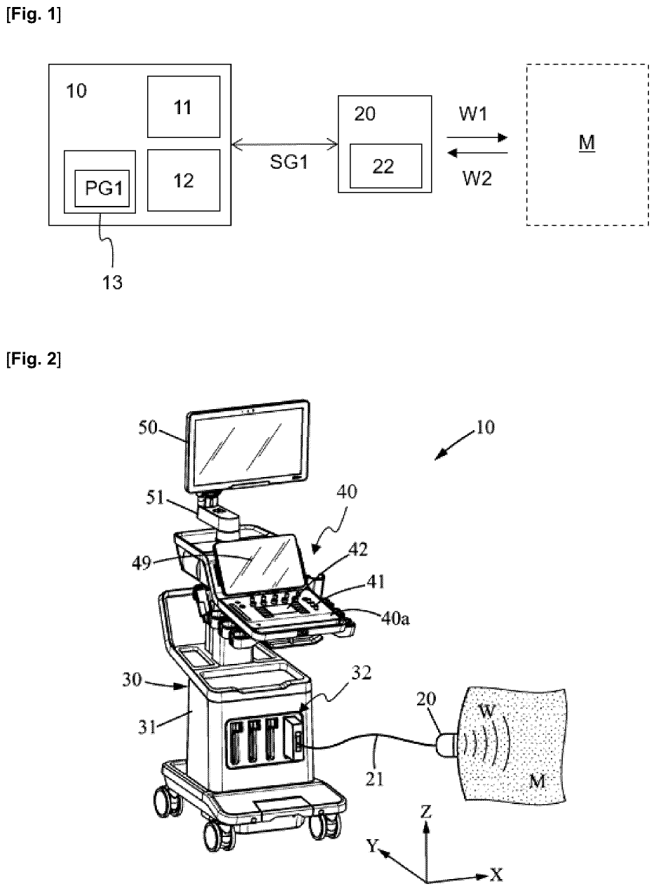

According to one example, the processing unit 11 and/or the control unit 12 are included in the body 31 (central element) of the system 10 represented for illustrative purposes in FIG. 2.

In particular, the processing unit 11 can be configured to control the electrical signals SG1 generated by the control unit 12. The processing unit 11 can also be configured to process (and/or interpret) electrical signals SG1 potentially received by the control unit 12 from the transducer device 20. These signals SG1 are representative of waves W2 received by the transducer device 20 from the medium M (FIG. 1). These waves W2 form, for example, one or more ultrasonic echoes, i.e. a response from the medium M to the ultrasound waves W1 transmitted towards said medium. The processing carried out by the processing unit 11 on the received signals SG1 may vary from case to case and comprise, for example, at least one of the operations of amplification, filtering, digitisation and conditioning of the signals SG1.

The system 10 can comprise the transducer device 20. Alternatively, the transducer device 20 can be external to the system 10. For example, the transducer device 20 may be connected to the system 10 by a cable or may communicate wirelessly with it. In the latter case, the transducer device 10 may, for example, comprise a battery and receive communication signals from the system 10, which represent the electrical signals SG1 (for example the control frequencies and/or any information included in the electrical signals). The transducer device 20 can therefore reproduce the electrical signals SG1 internally from the communication signals received.

The transducer device 20 can, for example, be a conventional wave-transmitting device. A difference according to the present invention may thus lie in the way in which the transducer device 20 is controlled and the associated means that are implemented to achieve such control. According to one variant, the transducer device 20 comprises a controller as described hereinafter in examples.

The system 10 can be stationary or mobile. The transducer device 20 can also be stationary or mobile. For example, the system 10 can be a fixed system (for example comprising a processing unit and a display device, as described below) and the transducer device 20 can be mobile (for example a sensor device, a measuring device, or a probe). Nevertheless, it is also possible that the transducer device 20 is integrated into the system 10, and that the system 10 is a mobile system. For example, the system 10 can be configured to be controlled autonomously, for example thanks to an included battery. Other examples are described below.

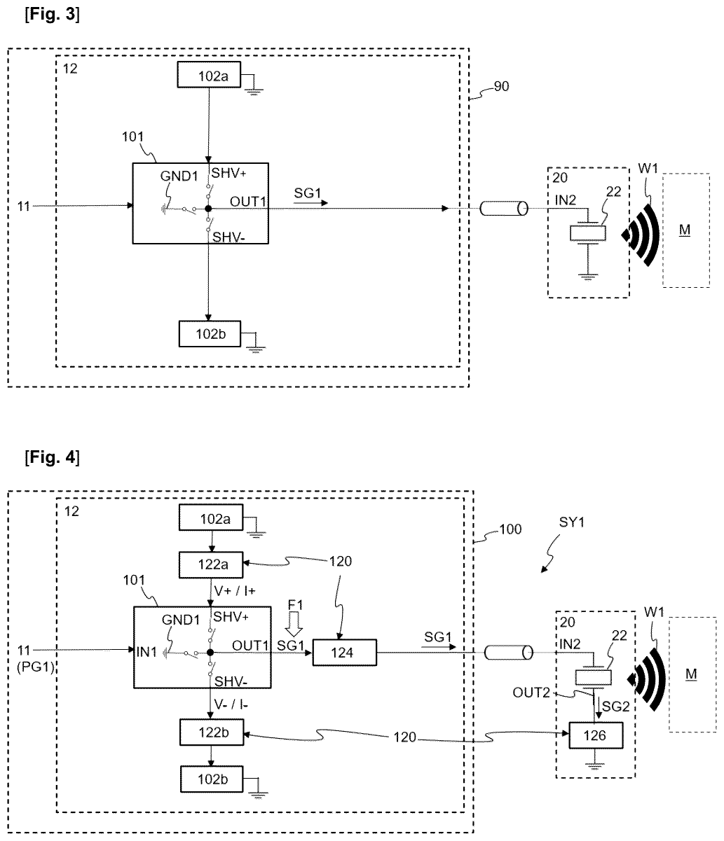

According to one example, the system 10 can comprise at least one memory 13 used by the processing unit 11 to control the control unit 10. This memory may optionally be part of the processing unit 11. In examples, the processor and the memory of the processing unit 11 can be incorporated into the system 10 illustrated in FIG. 3 or can be incorporated into a computer or a computer device communicatively linked to it. The memory can store instructions contributing to the implementation of the methods described in the present document in the form of a computer program PG1. This memory can in particular store instructions for processing ultrasound data and/or instructions for creating illustrative images of the medium observed.

According to the configuration and the type of computer device considered, the memory 13 can be volatile (such as RAM), non-volatile (such as ROM, flash, EEPROM, etc. or any other storage device and/or computer-readable medium as described below) or a combination of both. The memory 13 can, for example, be managed in DMA (“Direct Memory Access”) mode. The memory 13 used by the processing unit 11 can, for example, comprise all or part of a graphics card memory (or video card), this type of memory being in particular suitable for processing and/or sending image data that can be used to display one or more images on a display screen (or unit).

More generally, the system 10 can comprise storage devices (removable and/or non-removable), including, but not limited to, magnetic or optical disks or tapes.

In addition, the system 10 can comprise one or more input devices such as a keyboard, a mouse, a pen, a voice input, etc. and/or one or more output devices such as screens, loudspeakers, a printer, etc. The environment can also comprise one or more communication connections, such as LAN, WAN, point to point, etc. In certain embodiments, the connections can be used to establish point-to-point communications, wired communications, wireless communications, etc.

The system 10 can also comprise at least one computer-readable medium, such as the memory 13 in particular. The computer-readable media can be any available medium that the processing unit 11 (in particular its processor(s)) or other devices comprising the operating environment can access. By way of example, and without limitation, the computer-readable media may comprise computer storage media and communication media. The computer storage media comprise removable and non-removable, volatile and non-volatile media, implemented in any information storage technology or method such as computer-readable instructions, data structures, program modules or other data. The computer storage media do not comprise the communication media.

The system 10 can be a single computer operating in a network environment using logical connections with one or more remote computers. The remote computer can be a viewing station, a personal computer, a server, a router, a network PC, a peer device or another shared network node, and can generally comprise a plurality or all of the elements described above as well as others not mentioned. The logical connections may include any method supported by the communication media available.

As shown in FIG. 1, the ultrasound probe 20 can comprise, for example, one or more transducers (also known as transducer elements) with reference number 22, each being configured to convert an electrical signal SG1 received from the system 10 into ultrasound waves (and possibly also vice versa). The transducers 22 can thus be configured to transmit waves (or ultrasonic pulses, or ultrasound beams) W1 into the medium M, which corresponds to a transmission operation.

The transducers 22 can possibly also be configured to receive ultrasonic signals W2 from the medium M in a receiving operation, for example as a response to the transmitted waves W1. These received signals W2 can take the form of a set of backscattered echo signals as a response to the previously transmitted signals W1. Each transducer 22 can, for example, convert a received echo signal W2 into an electrical signal. The signal can subsequently be processed by the control system 10 (for example by the processing unit 11) or by any associated (dedicated) system, which is directly connected or not. For example, the signal W2 can be amplified, filtered, digitised and/or a signal conditioning operation can be carried out. The transducer elements may be disposed as a line of transducers, a matrix, or as an array of transducers or any other configuration.

It should be noted that the same transducer(s) 22 can be used to transmit waves (or pulses) W1 and, where appropriate, receive the waves W2 forming the response from the medium M, or different transducers can be used for transmitting and receiving waves. There can be one or more transmitting transducers and possibly a plurality of receiving transducers. In another variant, a single transducer 22 can be used. The transducers 22 can comprise piezoelectric crystals and/or other components that may be configured to generate and/or record and/or receive signals. In the interests of simplicity, it is considered that the transducers 22 in the following exemplary embodiments are piezoelectric transducers, without this specification being in any way limiting.

Various arrangements of the transducer(s) 22 are possible. For example, an array of transducers 22 comprising a plurality of transducers can be used. For example, a linear array can be provided comprising a plurality (for example between 2 and 10000) transducer elements juxtaposed along an axis X (horizontal direction or direction of the array X). In one example, the matrix is adapted for two-dimensional imaging (2D) of the medium M, but the matrix could also be a two-dimensional matrix adapted for 3D imaging of the medium M. A matrix of transducers 22 can consequently be used. However, it is also possible that the system comprises a single row of mobile transducers in a probe such that 3D imaging can be carried out. It is also possible that the array of transducers comprises one or more rows of transmitting transducer elements and one or more rows of receiving transducer elements. The array of transducers can also be a convex array comprising a plurality of transducer elements aligned along a curved line (for example in a curved probe).

According to one example, the processing unit 11 is configured to receive data from the transducer device 20, process data and/or send data to an external device, such as a processing device, a storage device, a display device, a server, a computer on which an artificial intelligence algorithm (IA) is executed, a dedicated workstation, or any other external device.

The system 10 can be an imaging system, for example in the medical field. The images generated by the system can either be analysed in real time, for example by a computer or algorithm, and/or an artificial intelligence module, or analysed later and/or in a different place from where the system 10 is located.

The system 10 can be a medical system, an ultrasound system or a medical ultrasound system. As a consequence, the device 20 can be a (or at least partially be part of a) medical and/or ultrasound probe.

For example, the system 10 can be associated with an ultrasound probe 20, in order to study a medium M (FIG. 1), in particular to collect ultrasound data from such a medium M. The medium M thus observed can be different from case to case. For example, it can be gravel, metal or living tissue, in particular human or animal tissue. It is also possible to observe a medium M comprising one or more mineral structures, for example (gravel, volcano, mapping of a ground, for example seabed, etc.).

The system 10 can thus be any type of electronic system. For example, the system 10 can be a type of medical system other than an ultrasound imaging system. Consequently, the transducer device 20 can be any type of imaging device or sensor, using waves other than the ultrasound waves (for example, waves having a wavelength different from the wavelength of the ultrasounds and/or waves that are not sound waves).

According to other examples, the system 10 and/or the transducer device 20 are configured for communication, imaging or scanning purposes, for example in the field of medical imaging, radar, sonar, seismology, wireless communications, radio astronomy, acoustics and biomedicine.

Examples of medical imaging systems comprise an ultrasound imaging system, an X-ray imaging system (for example system for performing mammograms) and an MRI (Magnetic Resonance Imaging) system.

FIG. 2 schematically shows non-limiting exemplary embodiments of the system 10 as previously described with reference to FIG. 1, namely an ultrasound imaging system in the examples under consideration.

The ultrasound imaging system 10 as shown in FIG. 2 can comprise:
an ultrasound probe 20 (corresponding for example to the transducer device 20 of FIG. 1),
a processing unit 30 configured to process or generate an image on the basis of the signals SG1 received by the probe 20 (this unit 30 corresponding for example to the processing unit 11 in FIG. 1),
a control panel 40 associated, linked or connected to the processing unit 30, said control panel being able to comprise at least one of keys 41 and a touch pad 42, and
one or more screen 50 configured to display the image or images generated by the processing unit 30.

The probe 20 can be connected to the processing unit 30 by a suitable connection means such as a cable 21 or a wireless connection. The probe 20 is also capable of transmitting ultrasound waves W into a medium M, and possibly also of receiving ultrasound waves W from the medium M, said received ultrasound waves may then be the consequence or result of reflections of said transmitted ultrasound waves on scattering particles inside said medium.

The probe 20 can be (or comprise) an array of transducers comprising a plurality of transducers (not shown), each converting an electrical signal SG1 into a vibration and vice versa. A transducer (also called a transducer element) is, for example, a piezoelectric element 22 as represented in FIG. 1. The array of transducers can, for example, comprise 128, 256 transducers or more. The array of transducers can be linear or curved and can be disposed on an outer surface of the medium M so as to be coupled to the medium and vibrate and transmit or receive ultrasound waves W.

The processing unit 30 can comprise receiver devices (not shown), comprising (or being), for example, receiver circuits, configured to process (for example amplify and/or filter) the signals SG1 received from the probe 20. These receiver devices can, for example, comprise converters for converting the received signals into data representing the signal (for example, analogue-to-digital converters (ADC) configured to convert a voltage into a digital code). The data obtained can be processed in various ways; it can in particular be temporarily stored in a memory accessible to the processing unit or processed directly in order to calculate intermediate processed data (“beamformed data”). The processing unit 30 can implement any known processing method to generate and/or process one or more images or maps or data on the basis of the signals received from the probe, such as the beam formation.

The processed data can be associated with different types of ultrasound images, which can be:
a B-mode image of the medium (image in B mode) generally displaced in greyscale to show the organs within the medium, and/or
a so-called Doppler image, showing the movements of fluids in the observed medium, for example the speed of movement and/or flow of fluids in the medium (image often using colour codes), which is useful, for example, to show blood vessels and their activities in the medium, and
an image showing a mechanical feature of the medium (elasticity), which is useful, for example, for identifying zones of different hardness that may turn out to be tumours within the medium (for example ShearWave™ Elastography image data).

The display screen 50 (for example mounted on a support arm 51) can be a screen for displaying the image processed by the processing unit 30 and/or can display various information depending on use, in particular help information or contextual gestural assistance that is adapted or can be adapted using the touchpad 42.

The control panel 40a constitutes all or part of a user interface that can be used by a user for interacting (controlling, receiving information, etc.) with the system 10.

A first exemplary embodiment of the system 10, hereinafter with reference number 90, is now described with reference to FIG. 3. This system 90 does not implement the concept of the present invention.

More specifically, the control system 90 comprises at least one pulser 101 configured to control a transducer 22 of the wave-transmitting device 20. To this end, the pulser 101 is configured to generate and send output signals SG1 to the transducer 22. These electrical signals SG1 are thus transmitted from the output OUT1 of the pulser to the input IN2 of the transmitting device 20, causing the transducer 22 to transmit ultrasound waves W1 towards the medium M, as already described. Although the system 10 can comprise a plurality of pulsers 101, it is assumed, for the sake of simplicity, that a single pulser 101 is used in this example.

As shown in FIG. 3, the control unit 12 comprises voltage regulators 102a and 102b respectively connected to the positive SHV+ and negative SHV− supply terminals of the pulser 101. In a known manner, these regulators 102a and 102b (also called PSUs for “Power Supply Unit”) are configured to supply a static voltage (or constant voltage) to the supply terminals SHV+/SHV− of the pulser 101. These regulators can be programmed to set a supply voltage for a given operating mode or probe 20. These regulators 102a, 102b thus supply power to the pulser 101, which is fixed over time. In practice, such voltage regulators can be implemented, for example, in a power board of the control unit 12.

However, the design of the system 90 in FIG. 3 has the same problems and shortcomings as those previously described (failures, lack of reliability, inadequate performance, etc.), due in particular to poor control of the output signals SG1 sent by the pulser 101 to the transmitting device 20.

Exemplary embodiments of the control system 10 (FIG. 1), subsequently with reference number 100, which implement the concept of the present invention, will now be described hereinafter with reference to FIGS. 4-15. Unless otherwise stated, the elements described above with reference to the control system 10 are applied in a similar manner to the control system 100. As already mentioned, it is assumed below that the system 100 is an ultrasound imaging system although other implementations are possible. For the sake of simplicity, certain elements described above with reference to FIGS. 1-3 are not described again in detail below.

According to one example, the control system 100 is positioned at the back of the probe connectors, for example behind the connectors 32 shown in FIG. 2. According to one example, the control system 100 is positioned at the interface between the cable 21 and the probe 20, or in the probe 20 itself.

More specifically, FIG. 4 schematically shows the control system 100 according to one example comprising a control unit 12 configured to control a transmitting device 20, namely an ultrasonic transducer device in the examples below. The control system 100 and the transducer device 20 together form a system with reference SY1.

As already mentioned, the control unit 12 comprises one or more pulsers 101 configured to control the transducer device 20 by means of electrical signals SG1. The electrical signals SG1 generated by the pulser(s) 101 are sent to the transducer device 20 to cause the transmission of ultrasound waves W1 into the medium M. The electrical signals SG1 thus generated by the pulser 101 are representative of the ultrasound waves W1 send to excite the medium M.

The transducer device 20 can comprise one or more ultrasonic transducers 22 (for example piezoelectric ones), each transducer being controlled by the signals SG1 from a respective pulser 101 of the system 100.

As illustrated in FIG. 4, each pulser 101 can use an electrical connection to exchange electrical signals SG1 with the transducer device 20, for transmission and possibly also for reception. By way of example, the system 100 can comprise 256 pulsers 101 configured to control respective transducers 22 of the transducer device 20.

The electrical connections used to transmit the output signals SG1 to the transducer device 20 can be (or comprise) electrically conductive physical links (or connections), the arrangement of which can vary case by case. Each connection LN can be formed, for example, in whole or in part by at least one electric track, for example one or more electric tracks formed on an electronic board (for example the control board 32 shown in FIG. 2). Each connection may possible comprise at least one portion of a cable or an electric cable (for example coaxial cable), such as the cable 21 shown in FIG. 2. According to one example, such an electrical connection can comprise at least one portion of electric track, and possibly also at least one portion of electric cable.

Unless otherwise stated, for the sake of simplicity in the invention, it is assumed below that the control system 100 uses a single pulser 101 to control a transducer 22 of the transducer device 20 even though variants are possible in which a plurality of pulsers 101 are used to control one or more transducers 22 of the device 20. The embodiments of the present invention apply in a similar manner to the control of one or more transducers 22 by a plurality of pulsers 101.

According to one example, the control unit 12 is also able to receive electrical signals SG1 coming from the transducer device 20 following excitation induced by the transmitted waves W1. These signals SG1 are representative of waves W2 (FIG. 1) received by the transducer device 20 from the medium M. These waves W2 form, for example, an ultrasonic echo, i.e. a response from the medium M to the ultrasound waves W1.

The pulser 101 (and more generally the control unit 12) is controlled by the processing unit 11. The latter can in particular control the pulser 101 to generate appropriate output signals SG1 according to the application under consideration. As shown in FIG. 1, the processing unit 11 may comprise at least one processor and a non-volatile memory 13. The control system 10 is thus configured to implement a control method for controlling the transducer device 20 as described below in specific embodiments. To this end, the system 10 can comprise a computer program PG1 stored in the non-volatile memory 13, this computer program PG1 comprising all or part of the instructions for implementing said method. The processor is configured to execute in particular the instructions defined by the computer program PG1.

The non-volatile memory 13 (FIG. 1) can correspond to any storage device (removable and/or non-removable) such as that described above in reference to the system 10. The control device 60 can also comprise at least one computer-readable medium as previously described in reference to the system 10 (for example computer storage media and/or communication media).

Similarly to the system 90 (FIG. 3), the control system 100 shown in FIG. 4 comprises voltage regulators 102a and 102b configured to supply electricity to the pulser 101 at its supply terminals, namely its positive supply terminals SHV+ and SHV− respectively. To do this, the regulators 102a and 102b (also called PSUs) supply a static voltage or constant voltage (also called supply rail) to supply power to the pulser 101 at its supply terminals.

However, the control system 100 (FIG. 4) differs from the system 90 (FIG. 3) in that it also comprises at least one controller 120, also known as a modulation block (or unit), configured to modulate the output signals SG1 transmitted from the pulser 101 to control the transducer 22. In other words, the controller(s) 120 are arranged in the system 100 (namely in the control unit 12 and/or in the transducer device 20 in the examples under consideration) to modulate F1 the output signals SG1 generated at the output OUT1 of the pulser 101 and sent to the input IN2 of the transducer 22.

The number of controllers 120 as well as their implementation can be adapted case by case. Whatever form the controller 120 takes, its function is to modulate (or control over time) the output signals SG1 sent by the pulser 101 to the transducer device 20. The system 100 can thus include a single controller 120 or a plurality of controllers 120 to carry out modulation F1.

In the present invention, the notion of modulation covers all modifications over time of at least one characteristic of a signal, such as its power, its voltage, its current, its frequency spectrum, its rise time and/or its fall time.

Modulation over a given period of a signal, such as the SG1 output signals emitted from pulser 101, implies, however, that said signal is not zero over the period considered. In other words, modulation in the sense of the present disclosure excludes the case where said modulated signal is zero (set to zero). Modulation in the sense of the present disclosure aims to modify a non-zero signal over time.

Implementation examples of the controller(s) 120 are described here below in reference to FIG. 4. In particular, the system 100 can comprise any one, or a plurality, of the following controllers 120 (FIG. 4):
a supply controller 122a and/or 122b;
an output controller 124; and
a return controller 126.

According to one example, the control system 100 comprises the supply controllers 122a and/or 122b.

According to one example, the control system 100 comprises the output controller 124.

According to one example, the control system 100 comprises the return controller 126.

According to one example, the control system 100 comprises the supply controllers 122a and/or 122b, and the return controller 126.

According to one example, the control system 100 comprises the supply controllers 122a and/or 122b, the output controller 124 and the return controller 126.

More specifically, according to one example, the controller 120 comprises one (or at least one) supply controller 122a and/or 122b, connected to a supply terminal of the pulser, namely to the positive supply terminal SHV+ and/or the negative supply terminal SHV− in the present case (FIG. 4). The system 100 can thus comprise only the positive supply controller 122a, or only the negative supply controller 122b, or both. The supply controllers 122a and 122b are configured to respectively control over time (or modulate) a supply voltage V+ and V− of the pulser 101.

The installation of a supply controller 122a/122b advantageously involves an implementation of limited complexity, and thus an easy implementation, for modulating the output signals, in particular because the supply controller is referenced to a fixed positive or negative voltage (that supplied by the regulators 102a/102b), and not to a variable bipolar voltage as is the case for the output controller 124, for example, which receives the voltage supplied at the output by the pulser 101.

Thanks to their position at the supply terminals of the pulser 101, the supply controllers 122a and 122b are particularly effective in controlling the signals supplied by the regulators 102a and 102b respectively. This makes it possible in particular to protect the regulators 102a and 102b, for example in the event of a short-circuit or overcurrent downstream of the supply controllers 122a and 122b.

It is possible to use only one of the two supply controllers, namely either 122a or 122b, for example if you wish to control the symmetry of the output signal SG1 of the pulser 101. For example, it is possible to modulate the positive part (or negative part, respectively) of the output signal SG1 as a function of what has been previously generated by the negative part (or positive part, respectively) of said signal. It is thus possible, for example, to obtain an output signal SG1 generated by a negative (or positive) sub-circuit of the pulser 101 during a first period, then to adapt the output signal SG1 during a second period (subsequent to the first period) to make it symmetrical and/or compensate for (or adapt) certain signal characteristics obtained during the first period.

According to one example, the pulser 101 comprises a ground terminal GND1, which is considered a supply terminal and can be connected to a supply controller similar to the controllers 122a and 122b for controlling over time a supply voltage at this ground terminal.

In general, a supply controller within the meaning of the present invention can therefore be connected to any one, or a plurality, of: the positive supply terminal SHV+, the negative supply terminal SHV− and the ground terminal GND1 of the pulser 101.

According to one example, the supply controller 122a connected to the supply terminal SHV+ is configured to modulate a power supplied to the pulser 101 on its supply terminal SHV+ from a static voltage supplied by the voltage regulator 102a. Likewise, according to one example, the supply controller 122b connected to the supply terminal SHV− is configured to modulate a power supplied to the pulser 101 on its supply terminal SHV− from a static voltage supplied by the voltage regulator 102b.

As already mentioned with reference to the system 90 (FIG. 3), the regulators 102a and 102b of the system 100 (FIG. 4) can be programmed or configured to supply a static (or constant) voltage over time, this voltage being, for example, fixed depending on the operating mode or the probe 20 used. In this example, the supply controllers 122a and/or 122b, supplied with voltage by the regulators 102a and/or 102b respectively, are configured to modify over time the power supplied to the pulser 101.

In one example, the controller 120 comprises (or is) an output controller 124 connected to the output of the pulser 101, i.e. to the output terminal OUT1 intended to output the output signals SG1 for controlling the transducer 22 of the transducer device 20. The output controller 124 is therefore configured to control over time (or modulate) the output signals SG1 sent to the transducer device 20 for generating the ultrasound waves W1 towards the medium M.

The installation of an output controller 124 advantageously makes it possible to limit the output current and/or the output voltage of the pulser 101, in particular so as not to stress or damage the pulser 101 and/or the transducer 22, insofar as the pulser 101 controls the transducer 22. As the output controller 124 is positioned between the pulser 101 and transducer 22, and thus as close as possible to the transducer 22, it is in particular able to effectively control the output signals SG1 transmitted by the pulser 101 and sent to the transducer 22.

According to one example, the output controller 124 is configured to modulate at least one of the electrical characteristics of the output signals SG1 of the pulser 101 out of voltage (V), current (I) and power (P) of said output signals SG1.

According to one example, the controller 120 comprises (or is) a return controller 126 connected to a return terminal OUT2 of the transducer 22 of the device 20. The return controller 126 is therefore configured to control over time (or modulate) return signals SG2 from the transducer 22.

More specifically, each transducer 22 of the transducer device 20 can, for example, form an electric dipole comprising an input terminal IN2 for receiving the output signals SG1 from a pulser 101 and a return terminal (or output terminal) OUT2 connected to a reference ground (also known as the “return path”). This return terminal OUT2 of the transducer 22 can, for example, be connected in series to a ground of the transducer device 20 or to any other reference ground of the control system 100 (or more generally of the system SY1), or connected in series to another electrical component (for example another pulser) of the control system 100.

By installing a return controller 126 connected in series to the return terminal OUT2 and to the reference ground, it is thus possible to modulate the power of the output signals SG1 transmitted from the pulser 101 to control the transducer 22.

The installation of the return controller 126 advantageously involves an implementation of limited complexity, and thus an easy implementation, for modulating the signals at the output of the transducer 22, and therefore consequently the output signals SG1 from the pulser 101. If the return controller 126 is connected in series with the return terminal OUT2, and is thus close to the transducer 22, the return controller 126 is particularly effective in controlling the signals at the output of the transducer 22 and thus in protecting the transducer 22.

The system 100 can comprise only one of the controllers 122-126 or any combination of these controllers 120 (for example the controllers 122a, 122b and 124). For the sake of simplicity, it is assumed below that the system SY1 comprises controllers 120, for example the controllers 122a, 122b, 124 and 126 as previously described, to modulate the power of the output signals SG1 from the pulser 101 intended to control the transducer 22.

It is possible to use only one controller out of the previously described controllers 122a, 122b, 124 and 126, for example, only controller 122a, only controller 122b or only controller 126. The supply controllers 122a, 122b advantageously allow the current and/or the supply voltage of the pulser to be limited, in particular so as not to stress/damage in particular the pulser 101. The output controller 124 and/or the return controller 126 advantageously allow the current and/or the voltage applied to the transducer 22 to be limited, in particular so as not to stress/damage in particular the transducer 22.

Various implementations of these controllers 120 are possible to enable modulation F1. By way of example, the controller(s) 120 can be implemented in the form of one or more discrete and/or integrated components, for example an integrated circuit. The use of discrete components enables in particular robust, dense mounting, for example in an electronic board.

According to one example, the controllers 120 take the form of an integrated circuit, for example an FPGA (field-programmable gate array).

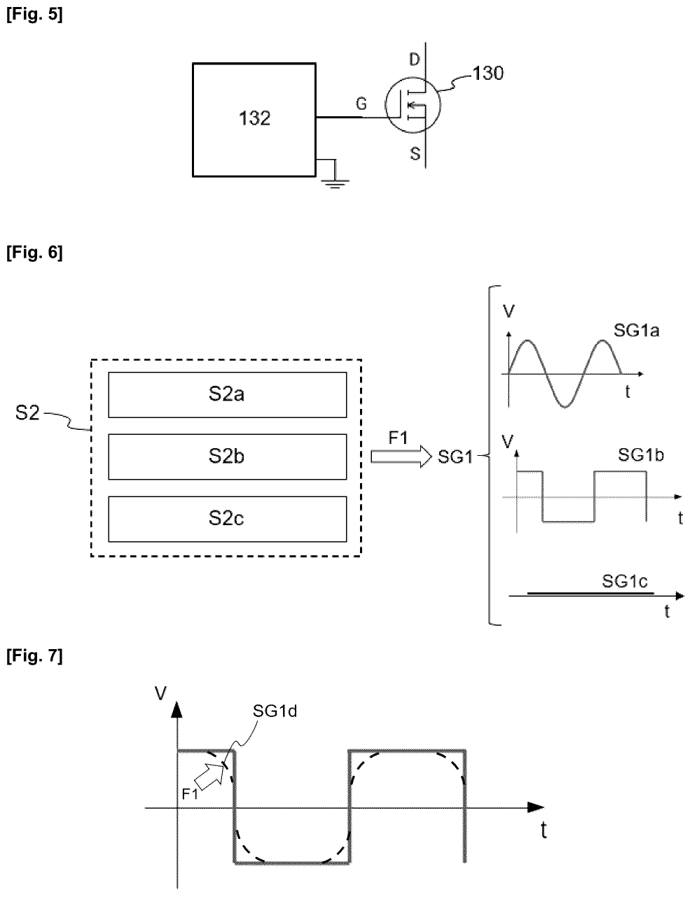

FIG. 5 schematically show an exemplary implementation of a controller 120 according to the present invention. As shown, the controller 120 takes the form of a MOSFET transistor (field-effect transistor with an insulated gate) in this example, the gate voltage of which is modulated over time, for example by means of a control unit comprising a DAC (Digital to Analogue) converter with reference number 132.

To do this, the converter 132 is configured to send commands to the MOSFET gate G. In the case of a supply controller 122a/122b, either one of the drain (D) and source (S) terminals can be connected to the regulator 102a/102b respectively and the other to the corresponding supply terminal SHV+/SHV− of the pulser 101. Similarly, in the case of the output controller 124, either one of the drain (D) and source (S) terminals can be connected to the output terminal OUT1 of the pulser 101 and the other can be connected in series to the input IN2 of the transducer 22.

The example in FIG. 5 advantageously enables the controllers 120 to be implemented with limited complexity and a low cost of implementation. This makes it easier to develop (design), implement and maintain the wave-transmitting device. More complex implementations are nevertheless possible.

A control method implemented by the control system 100 as previously described (FIGS. 4-5) is now described in conjunction with FIGS. 6-9 according to specific embodiments. To this end, the control system 100 (or more generally the system SY1) can run the computer program PG1. The system 100 can execute instructions from at least one computer program, including for example the program PG1.

During step S2 of modulation, the output signals SG1 from the pulser 101 are modulated by one (or at least one) controller 120. This modulation—with reference F1—can be performed by a controller 120 according to at least one of:
a) control (S2a) of a supply voltage (V+ and/or V−) of the pulser 101 by a supply controller 122a and/or 122b connected to a supply terminal IHV+ and/or IHV− of the pulser 101;
b) control (S2b) of output signals SG1 by an output controller 124 connected to the output OUT1 of the pulser 101; and
c) control (S2c) of return signals SG2 from the transducer 22 by a return controller 126 connected to a return terminal OUT2 of the transducer 22.

In other words, modulation F1 is performed by means of any one, or several, of the abovementioned controllers 120. This modulation F1 makes it possible to control over time the output signals SG1 emitted from the pulser 101 in order to control the transducer 22. In particular, the power and/or shape of the signals SG1 sent to the input IN2 of the transducer 22 can be adapted or modified over time.

According to one example, the modulation F1 causes a variation over time of at least one characteristic of the output signals SG1, this characteristic taking at least two distinct non-zero values during said modulation.

According to one example, the power supply controllers 122a and/or 122b are used to perform the modulation F1 of the output signals SG1.

According to an example, the return controller 126 is used to perform the modulation F1 of the output signals SG1.

According to one example, the power supply controllers 122a and/or 122b are used in combination with the return controller 126 to perform the modulation F1 of the output signals SG1.

According to one example, the shape and/or amplitude of the voltage transmitted to the transducer 22 are modulated (or adapted). Consequently, for a given impedance of the transducer 22 (or more generally of the probe), the current and power transmitted are consequently modified.

Modulation F1 makes it advantageously possible to improve the control of the transducer device 20 and thus the performance and reliability of the system SY1 overall. The modulation F1 makes it possible in particular to effectively and flexibly control the output signals SG1 sent to the transducer 22 and thus to ensure that the theoretical limit values of the parameters (in particular in current, voltage and/or power) of the transducer 22, and more generally of the transmission chain from the pulser 101 to the transducer 22, are respected. By controlling the transducer 22 more precisely, it is possible to avoid over-stressing the system, limit failures (faults, degradation, etc.), ensure a good level of reliability of the system and guarantee that the system has satisfactory performance.

Thanks to the modulation F1, it is in particular possible to adapt the waves sent with more flexibility (in power, frequency, duration, etc.) without impairing the pulser 101 or transducer 22, which makes it possible to overcome certain compromises or limitations (particularly in terms of power) usually associated with this type of device to guarantee a good performance and normal ageing of the whole unit. In contrast to conventional systems, the concept of the present invention makes it possible to configure the transducer device 22 so as to transmit the desired waves, i.e. in accordance with the desired wave characteristics for the intended application.

With limited modification to the whole unit, and thus a small additional cost, it is thus advantageously possible to obtain a transducer device 22 with high-level capabilities.

It is possible, for example, to improve the image quality, such as the quality of an ultrasound image, in an ultrasound imaging application, for example in the medical field, by improving the control of the transducer 22. Thanks to the method of the present invention, it is in particular possible to obtain a good image quality.

Various implementations of the modulation F1 of the output signals SG1 are possible. As shown in FIG. 6, the modulation F1 is, for example, configured to control (or adapt) the waveform of the output signals SG1. The modulation F1 is, for example, configured to cause a change in the waveform of the output signals SG1 compared to the waveform generated intrinsically at the output of the pulser 101 (i.e. without modulation F1). It is thus possible to adapt the waveform of the signals SG1 (and thus of the waves W1) for a given pulser 101, and thus make the control of the transducer 22 more flexible because it depends less on the type of pulser 101 used. It is in particular possible to adapt the waveform of the signals SG1 to the constraints and specificities of the various imaging modes that may be used (for example, B-mode, Doppler mode, ShearWave™ Elastography, etc.).

By way of example, modulation F1 can cause the generation, at the output of the pulser 101, of output signals SG1 of sinusoidal form (signal SG1a, FIG. 6), of square form, of intermediate or hybrid form (signal SG1d, FIG. 7), or of any other appropriate form depending on the application. Alternatively, modulation F1 can cause the deactivation of the pulser 101, which is equivalent to generating a zero output signal SG1 (signal SG1c, FIG. 6).

More specifically, as shown in FIG. 7, it is assumed, for example, that the pulser 101 is of the “pulse” type, i.e. a pulser intrinsically configured to generate square output signals SG1. According to one example, the modulation F1 performed by means of the controller(s) 120 causes a change in the waveform from the intrinsic square waveform to a modified waveform (or modulated waveform), i.e. a sinusoidal waveform or an intermediate (or hybrid) waveform SG1d between the square form and the sinusoidal form. By approximating the sinusoidal waveform, it is advantageously possible to limit the harmonics present in the signals SG1 transmitted to the transducer 22, which makes it advantageously possible to limit the heat dissipation and electromagnetic radiation, to improve the energy efficiency of the system and to extend the life of the system.

According to one example, the modulation F1 is carried out by means of at least two supply controllers 122a and/or 122b. The modulation F1 causes a variation over time of the respective supply voltages V+ and/or V− of the pulser 101, these voltages varying according to (or taking on) at least two distinct non-zero values during said modulation. In this way, the supply voltages V+ and/or V− vary according to a plurality of distinct, non-zero voltage values, while the pulser 101 generates non-zero output signals SG1. This makes it possible to create output signals SG1 of various shapes, offering greater flexibility and better control of the pulser 101. With a same pulser 101, the waveform of the output signals SG1 can be adapted by the modulation F1. For a given pulsed pulser 101 (intrinsically configured to deliver square output signals SG1), the modulation F1 can be used to generate sinusoidal output signals SG1 at the output of pulser 101, or signals of intermediate waveform between square and sinusoidal. In other words, by applying the modulation F1 to the supply voltages V+ and/or V− of the pulser 101, it is advantageously possible to transform a “pulsed” type pulser 101 into a linear type pulser 101, i.e. capable of delivering output signals SG1 of various shapes, in particular of sinusoidal or intermediate shape.

According to one specific example, the modulation F1 is configured to control (or adapt) the power of the output signals SG1 emitted from the pulser 101 in order to control the transducer 22.

According to one example, the modulation F1 is performed at a modulation frequency Fm greater than or equal to a pulse frequency Ft at which the transducer 22 transmits waves W1 in response to the output signals SG1 from the pulser 22. The modulation frequency Fm can, for example, be of the order of the pulse frequency Ft.

By way of example, the pulse frequency Ft is between 100 kHz and 20 MHz (or even between 1 MHz and 20 MHz for the emission of sound waves W1, for example in medical imaging applications). In this case, the modulation frequency Fm can be such that Fm ≥100 kHz (where 100 KHz is the lower limit of the pulse frequency).

According to one example, the modulation F1 is performed at a modulation frequency Fm greater than or equal to a fraction 1/P of the pulse frequency Ft, where P is an integer equal to 10, for example. The modulation frequency Fm can be lower than the pulse frequency Ft according to certain examples. In a case, for example, where a firing sequence of a few periods of a square signal is produced, this sequence can advantageously be modulated according to a Gaussian envelope (low amplitude at the start of the firing, maximum in the middle and low again at the end). In this case, the frequency Fm is less than the pulse frequency Ft, but must be greater than the maximum frequency of variation of the supply voltage supplied by the regulators 102a and 102b.

According to one example, the output signals SG1 are modulated in a frequency band with reference Bm. The transducer 22 (or more generally the transducer device 20) is characterised by a bandwidth in which it is able to convert output signals SG1 into ultrasound waves W1. The frequency band Bm is therefore configured so that its intersection with said bandwidth is not equal to zero.

The control system 100, and more generally the system SY1, can, for example, be implemented in medical imaging applications, in particular involving living beings (humans and/or animals).

According to one example, the output signals SG1 are modulated F1 continuously or discretely over time.

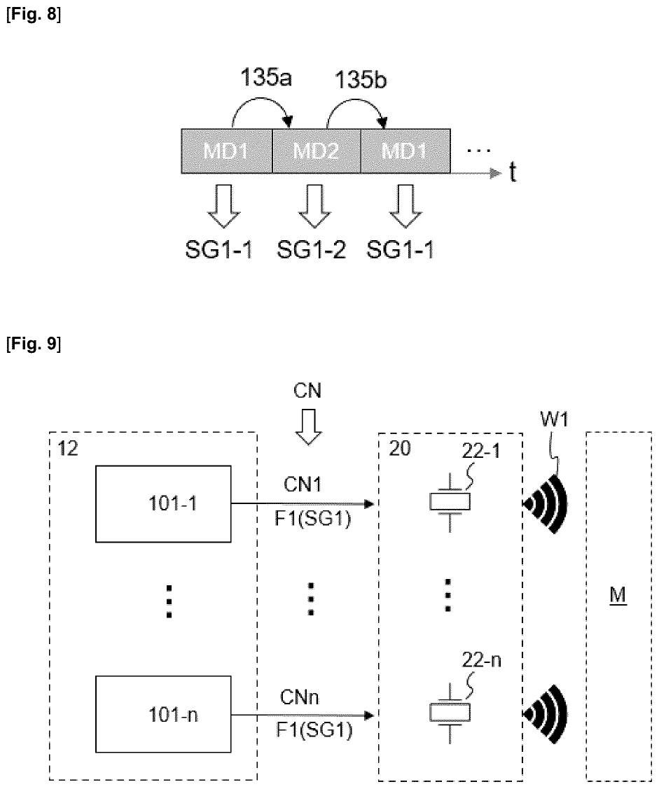

As shown in FIG. 8 according to one example, the modulation F1 performed by means of the controller(s) 120 can comprise switching 135a between two modes, i.e.:
a first mode MD1 causing the pulser 101 to emit output signals SG1 (with reference SG1-1) according to a first configuration of waveform and/or power; and
a second mode MD2 causing the pulser 101 to emit output signals SG1 (with reference SG1-2) according to a second configuration of waveform and/or power that is different from the first configuration.

By adapting the modulations carried out in modes MD1 and MD2, it is thus possible to flexibly control the transducer 22 and thus ensure good performance and a good level of reliability of the system. By way of example, the controller(s) 120 can be configured such that:
in the first mode MD1, no modulation F1 is carried out (deactivation of the controller(s) 120); the output signals SG1-1 are the signals intrinsically generated by the pulser 101 in the absence of any modulation; and
in the second mode MD2, modulation F1 is carried out (activation of the controller(s) 120) such that the output signals SG1-2 thus modulated are different from those emitted in the first mode MD1.

According to one example, in the second mode MD2, the output signals SG1-2 are constrained, i.e. limited in power compared to the intrinsic output signals such that the transducer 22 operates in reduced mode (transmission of waves W1 less powerful than in mode MD1 without modulation).

It is thus possible to configure the controller(s) 120 to switch 135a from mode MD1 to mode MD2 and/or to switch 135b from mode MD2 to mode MD1 (FIG. 8). To this end, it is possible, for example, to control the gate voltage on the gate G of the MOSFET 130 used as a controller 120 (for example 120a/120b, 122 and/or 124) by means of the converter 132 such as to switch as desired between MD1 and MD2.

It is, for example, possible to configure the controller(s) 120 to alternate between modes MD1 and MD2 (FIG. 8). The lengths of time that the modes MD1 and MD2 are active can be set as equal of different.

According to one example, the output signals SG1 are modulated F1 as a function of an imaging mode implemented by the pulser 101 to control the transmitting device 20.

According to one example, the output signals SG1 are modulated F1 as a function of a type of transmitting device 20.

According to one example, the output signals SG1 are modulated F1 as a function of user parameters of the transmitting device 20 (for example via software control, possibly using artificial intelligence).

According to one example, the output signals SG1 are modulated F1 as a function of at least one of the above criteria, i.e. an imaging mode implemented by the pulser 101, a type of transmitting device 20 and user parameters of the transmitting device 20.

As mentioned above, the concept of the present invention can be applied in a similar way to a plurality of pulsers 101 of the control system 100. Thus, according to one example shown schematically in FIG. 9, the transducer device 20 comprises a plurality of transducers 22 each controlled by a respective pulser 101. In this case, the output signals SG1 are modulated F1 independently per channel CN between each transducer 22 and the respective pulser 101. To do this, modulation F1 is carried out on the output signals SG1 of each pulser 101 by one (or at least one) controller 120 as previously described for a given pulser 101.

More specifically, each transducer 22 can be controlled by a respective pulser 101 via a transmission chain allowing output signals (or electrical signals) SG1 to be transmitted from the pulser 101 to the transducer 22. Each of these transmission chains constitutes a channel CN (FIG. 9) via which a pulser 101 can control a respective transducer 22 of the transducer device 20 and thus control the waves W1 transmitted by said transducer into the medium M. As shown by way of example in FIG. 9, n pulsers with references 101-1 to 101-n can thus be configured to control respective transducers 22-1 to 22-n via respective channels CN1 to CNn (n being an integer greater than or equal to 2). It is thus possible to carry out modulation F1 per channel and to thus independently adapt the output signals SG1 transmitted to each transducer 22.

This modulation F1 per channel advantageously makes it possible to control the transducer device 20 even more precisely and flexibly. This advantageously makes it possible in particular to adapt the output signals SG1 supplied to each transducer 22 independently of the other transducers 22. For example, as part of a calibration of the control system 100 or of the system SY1, the power transmitted to each transducer 22 can be optimised. If the same modulation impacts several channels/transducers at once, the calibration has to take account of the variations of characteristics of all of the transducers of the group in question and a compromise must therefore be found to respect the limits and specificities of each transducer. The modulation per channel advantageously makes it possible to fine-tune the calibration or configuration of each transducer separately.

It should be noted that it is possible to carry out such modulation F1 in all the channels (or transmission chains) of the system SY1 or only in a sub-section thereof, depending on the desired effect. It is thus possible to configure the various transducers 22 of the system flexibly, for example by modulating the output signals SG1 supplied to at least one transducer 22 whilst the output signals SG1 supplied to at least another transducer 22 are not modulated.

According to one example, it is, for example, possible to configure the modulation F1 per channel to control the transducers 22 of the transducer device 20 according to a function called apodisation.

Conventionally, such an apodisation function can be carried out by varying the amplitude of the output signals SG1 transmitted to the transducers 22 as a function of the position of the transducers relative to a focal point (a focus area) in the medium M, which makes it possible to adapt the energy supplied to the medium E and in particular to focus this energy more effectively on a point (or region) of interest in the medium M. Conventionally, with on-off/square pulsers, this variation in amplitude is achieved by modifying the duty cycle of the output signal of the pulser (ratio between the time spent in the “high” state (V+ or V−) and the period of the signal). This effectively changes the RMS value of the power transmitted to the transducer. However, modifying the duty cycle also modifies the spectral content of the transmitted signal, which limits the quality of apodisation, which only affects the amplitude of the signal and not its spectrum.

According to one example, the modulation F1 is configured to modify the amplitude of the signals SG1 transmitted as a function of the position of the transducer 22, while maintaining the same duty cycle between all the channels, which makes it possible to maintain the same spectral content for each transducer 22. The effect of apodisation is therefore better, particularly on image quality in the case of an imaging application, since the theoretical principle of apodisation is better respected.

According to one example of the present invention, the system 100 can be configured to carry out modulation F1 per channel on a plurality of transducers 22 of the transmitting device 20 such that the further away a transducer 22 of the device 20 is from a focal point of interest in the medium M, the lower the voltage of the output signal SG1 sent to this transducer 22 (same waveform but decreasing voltage). By controlling the modulation F1 per channel, it is advantageously possible to control the transducers 22 to transmit waves according to an apodisation function with more precision and flexibility. For example, it is possible to configure the modulation F1 so that at least one transducer 22 is voltage-limited (or even deactivated) in order to limit the energy supplied by this transducer to the medium M. It is thus possible to adapt the control of the transducers 22 and effectively avoid issues of poor focusing of the waves W1 and undesirable echoes W2.

In the control method described above with reference in particular to FIG. 6, the output signals SG1 from the pulser 101 can be modulated F1 in various ways, for example deterministically or in any way, for example by predefining the way in which the signals SG1 are to be modulated (without taking actual system performance into account) or by adapting the modulation F1 as a function of the behaviour or characteristics of the control system 100, or of the system SY1 in its entirety. In the exemplary embodiments below, the modulation F1 of the output signals SG1 is adapted as a function of the behaviour of the transmission chain extending from the pulser 101 to the transducer 22.

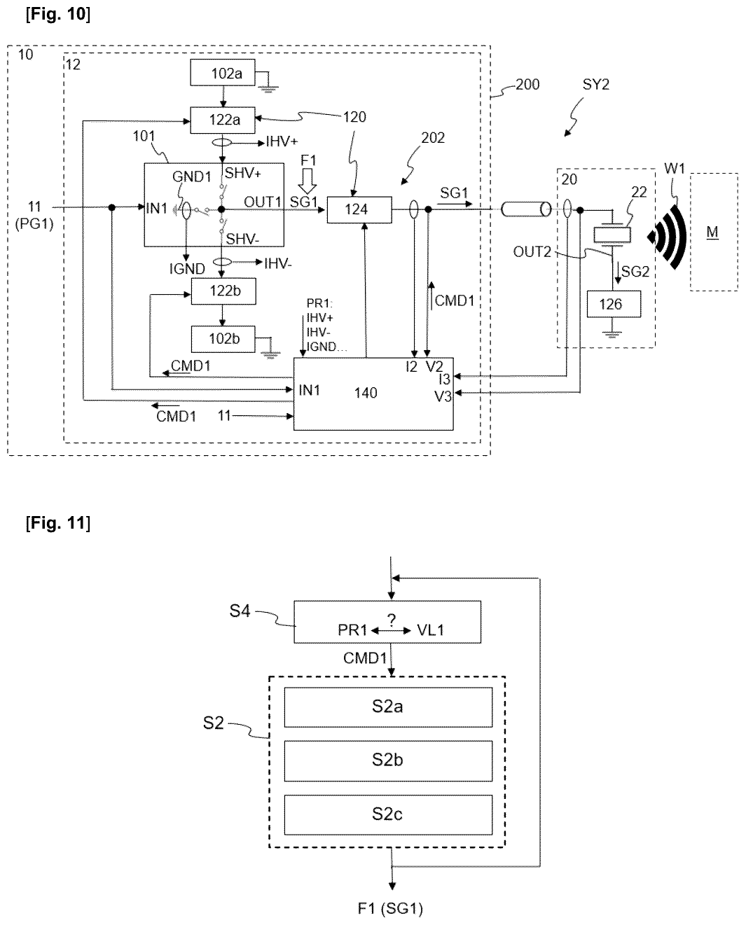

Exemplary embodiments of the control system 100 (FIGS. 4-9), subsequently with reference number 200, which implement the concept of the present invention, will now be described hereinafter with reference to FIGS. 10-15. Unless otherwise stated, the elements described above with reference to the control system 10 are applied in a similar manner to the control system 100. As already mentioned, it is assumed below that the system 200 is an ultrasound imaging system although other implementations are possible. For the sake of simplicity, certain elements described above with reference to FIGS. 1-9 are not described again in detail below.

Unless otherwise stated, for the sake of simplicity in the invention, it is assumed below that the control system 200 uses a single pulser 101 to control a transducer 22 of the transducer device 20 even though variants are possible in which a plurality of pulsers 101 are used to control one or more transducers 22 of the device 20. The embodiments of the present invention apply in a similar manner to the control of one or more transducers 22 by a plurality of pulsers 101.

More specifically, FIG. 10 schematically shows the control system 200 according to one example comprising the control unit 12 configured to control the transducer device 20, namely an ultrasound device in this example. The control system 200 and the transducer device 20 together form a system with reference SY2.

The control system 200 (FIG. 10) mainly differs from the system 100 (FIG. 4) in that the modulation F1 of the output signals SG1 emitted by the pulser 101 is adapted as a function of the behaviour of a transmission chain 202 extending from the pulser 101 to the transducer 22. To do this, the control system 200 comprises a control unit 140 configured to measure at least one operating parameter PR1 of the transmission chain 202 via which the pulser 101 sends the output signals SG1 to the transducer 22. The control unit 140 is also configured to control the controller(s) 122 included in the control system 200, or more generally in the system SY2, as a function of the parameter(s) PR1 measured. The control unit 140 is, for example, configured to send commands CMD1 to the controllers 120 to control the modulation F1 of the output signals SG1 over time. It is thus possible to improve the control of the transducer 22 by adjusting the modulation F1 to the behaviour of the system.

The transmission chain 202 comprises a set of hardware components of the system SY2, extending from the pulser 101 to the transducer 22 and possibly comprising at least one intermediate component or other participant in the transmission of the electrical signals SG1 from the pulser 101 to control the transducer 22. This transmission chain 202 comprises in particular the pulser 101 and the transducer 22, or even other components such as an electrical connection connecting the pulser 101 to the transducer 22.

This transmission chain 202 can be characterised by one or more operating parameters PR1, the number and nature of which can vary according to use.

The control unit 140 can in particular comprise, or use, any means appropriate for measuring the operating parameter(s) PR1 of the transmission chain 202. Examples of implementation of these measurement means are subsequently described with reference in particular to FIGS. 11-15.

According to one example, the operation parameter(s) PR1 measured by the control unit 140 characterise at least one of a pulser state and a transducer 22 (or more generally transducer device 20) state.

The operating parameter(s) PR1 measured can comprise at least one electrical parameter (voltage, current or power for example) and/or at least one physical parameter such as temperature, an electric field, a magnetic field or a sound pressure.

According to one example, the operating parameter(s) PR1 measured by the control unit 140 comprise at least one of:
a voltage V2 at the pulser 101 output;
a supply voltage (HV− and/or HV+) of the pulser 101;
a ground voltage of the pulser 101;
a voltage V3 at the terminals of the transmitting device 20;
a voltage received from the transducer 22;
a current I2 at the pulser 101 output;
a supply current (IHV+, IHV−) of the pulser 101;
a ground current IGND of the pulser 101;
a current I3 flowing through the transmitting device 20;
a temperature of the pulser 101;
a temperature of the transmitting device 20;
an electric field emitted by the pulser 101;
an electric field emitted by the transmitting device 20;
a magnetic field emitted by the pulser 101;
a magnetic field emitted by the transmitting device 20;
a sound pressure emitted by the transmitting device 20; and
a sound pressure received by the transmitting device 20.

The operating parameter(s) PR1 measured can correspond to any of the above parameters or to any combination of at least two of them.

The received voltage mentioned above refers to the voltage of a response signal, called an “echo” signal, generated by the transducer 22 in response to the waves W2 (FIG. 1) received from the medium M, for example in response to the waves W1 (stimuli) emitted by the transducer device 20.

A control method implemented by the control system 200 as previously described (FIG. 10) is now described in conjunction with FIG. 11 according to specific embodiments. To this end, the control system 200 (or more generally the system SY2) can run the computer program PG1. The system 100 can execute instructions from at least one computer program, including for example the program PG1 and/or one program (not shown) implemented by the control unit 140.

According to one example, the control unit 140 operates in response to instructions from the processing unit 11 (FIG. 10).

During step S4 of measurement (FIG. 11), the control unit 140 measures one or more operating parameters PR1 of the transmission chain 202 via which the pulser 101 sends the output signals SG1 to the transducer 22. It is assumed below that a plurality of parameters PR1 are measured although alternatives are possible where a single parameter PR1 is measured.

During step S2 of modulation, the output signals SG1 from the pulser 101 are modulated by one (or at least one) controller 120, as already described with reference to FIG. 6. In particular, as already described, this modulation F1 can be performed by a controller 120 according to at least one of:
a) control (S2a) of a supply voltage (V+ and/or V−) of the pulser 101 by a supply controller 122a and/or 122b connected to a supply terminal IHV+ and/or IHV− of the pulser 101;
b) control (S2b) of output signals SG1 by an output controller 124 connected to the output OUT1 of the pulser 101; and
c) control (S2c) of return signals SG2 from the transducer 22 by a return controller 126 connected to a return terminal OUT2 of the transducer 22.

The control method implemented by the system 200 (FIG. 10) differs from the one previously described with reference to the system 100 (FIG. 4) in that the output signals SG1 are modulated F1 (S2) as a function of the operating parameters PR1 measured in S4. In other words, the modulation F1 carried out in S2 is based on the result of the measurements carried out previously in S4. It is thus advantageously possible to adapt the signals SG1 emitted by the pulser 101 to control the transducer 22 as a function of the behaviour of the transmission chain 202, thus making the control more adaptive and therefore more efficient.

According to one example, the control unit 140 compares (S4) the operating parameters PR1 with respective threshold values, for example to determine if these parameters PR1 exceed said threshold values. The modulation F1 carried out in S2 can therefore be based on a result of these comparisons. This comparison can be carried out for one or a plurality of operating parameters PR1 depending on the case under consideration. It is thus possible to check whether the limits of various components (or parts) of the transmission chain 202 are respected (maximum voltage limit, maximum current limit, maximum temperature limit, etc.).

According to one example, the control unit 140 controls at least one supply controller 122a/122b such that:
the corresponding supply voltage is regulated to a first value during the control S2a (FIG. 11) if a (at least one) operating parameter PR1 does not exceed a respective threshold value VL1, and
the control S2a comprises adapting the supply voltage from the first value to a second value that is different from the first value if the (or said at least one) operating parameter PR1 exceeds said respective threshold value VL1. According to one example, the first and second values are non-zero values.

It is thus possible to adapt the supply voltage of the pulser 101, and therefore of its output signals SG1 sent to the transducer 22, as a function of the voltage level at the supply terminals of the pulser 101. If necessary, the power of the pulser 101 can, for example, be limited by defining a second value (not zero) lower than the first value (operation of the pulser 101 in reduced or limited mode). According to one example, the pulser 101 can be deactivated if the threshold value VL1 has been reached.

According to one example, the control unit 140 controls the controllers 120 to bring at least one measured operating parameter PR1 in line with a reference value (or setpoint), for example to align a power efficiency of the transmitting device 20 with a given efficiency setpoint (efficiency between the input power provided by the regulators 102a/102b and the output power provided by the transducer(s)).

According to one example, the output signals SG1 are modulated F1 (S2, FIG. 11) by slaving at least one of the controls S2a, S2b and S2c as a function of the operating parameter(s) PR1 measured in S4. It is thus possible to dynamically adapt the output signals SG1, for example in real time, so as to achieve reactive and efficient control of the transducer 22.

According to one example, the operating parameter(s) PR1 are measured S4 (FIG. 11) during a calibration of the transmission chain 202. This calibration 202 can, for example, be carried out only once (for example at the factory production stage) or repeatedly over time (for example when the control system 200 is started up or powered up, or each time a predefined number of uses of the control system 200 has been reached or each time a predefined duration of use of the control system 200 has been reached).

The modulation F1 is, for example, carried out after the aforementioned calibration, for example in response to at least one command CMD1 from the control unit 140. This command or these commands CMD1 can, for example, be determined as a function of the operating parameter(s). These commands CMD1 can be controlled or not over time as already described. It is thus possible to adapt the modulation F1 of the signals SG1, and thus to adjust the control of the transducer device 20, as a function of the overall behaviour. This makes it possible in particular to adapt the configuration of control systems to compensate for physical or structural disparities between components between systems, which makes it possible to ensure greater uniformity of performance and reliability between systems.

As already mentioned, the concept of the present invention can be applied in a similar way to a plurality of pulsers 101 of the control system 200. Thus, according to one example, the transducer device 20 comprises a plurality of transducers 22 each controlled by a respective pulser 101. In this case, the output signals SG1 are modulated F1 independently per channel between each transducer 22 and the respective pulser 101. To do this, modulation F1 is carried out on the output signals SG1 of each pulser 101 by one (or at least one) controller 120 as previously described for a given pulser 101. Each transducer 22 can thus be controlled by a respective pulser 101 via a transmission chain 202 allowing output signals (or electrical signals) SG1 to be transmitted from the pulser 101 to the transducer 22. Each of these transmission chains 202 constitutes a channel via which a pulser 101 can control a respective transducer 22 of the transducer device 20 and thus control the waves W1 transmitted by said transducer into the medium M. It is thus possible to carry out modulation F1 per channel as a function of at least one operating parameter PR1 measured during step S4 (FIG. 11) of measurement so as to independently adapt the output signals SG1 transmitted to each transducer 22.

According to one example, the control unit 140 measures (S4; FIG. 11) at least one operating parameter PR1 of transmission chains 202 via which each transducer 22 of the transducer device 20 receives output signals SG1 from a respective pulser 101. Upon detection that one (or at least one) operating parameter PR1 of a transmission chain 202 meets a predefined criterion (for example exceeding a threshold value VL1), the modulation F1 of the output signals SG1 emitted from the pulser 101 of the transmission chain 202 to the transducer 22 of said chain is adapted accordingly (for example, modification of a power and/or waveform configuration according to which the output signals SG1 are emitted). This controlled modulation F1 per channel makes it possible to control the transducer device 20 even more precisely and flexibly. The individual modulation per channel makes it advantageously possible to compensate for differences or variations in characteristics between channels or transducers in order to limit the disparities in behaviour between transducers and thus standardise their performance.

According to one example, upon detection of a change in voltage or current in a transmission chain 202 pointing to a malfunction or failure of the transducer 22 of said chain, the control unit 140 controls the controller(s) 120 to reduce or limit the power of the output signals SG1 emitted to control the transducer 22 of the transmission chain 202. It is thus possible to dynamically reconfigure a transducer 22 exhibiting an anomaly such that this transducer continues to transmit waves W1, but waves limited or constrained in power, towards the medium M.

Examples for implementing means for measuring the operating parameters PR1 are now described purely by way of illustrating the present invention.

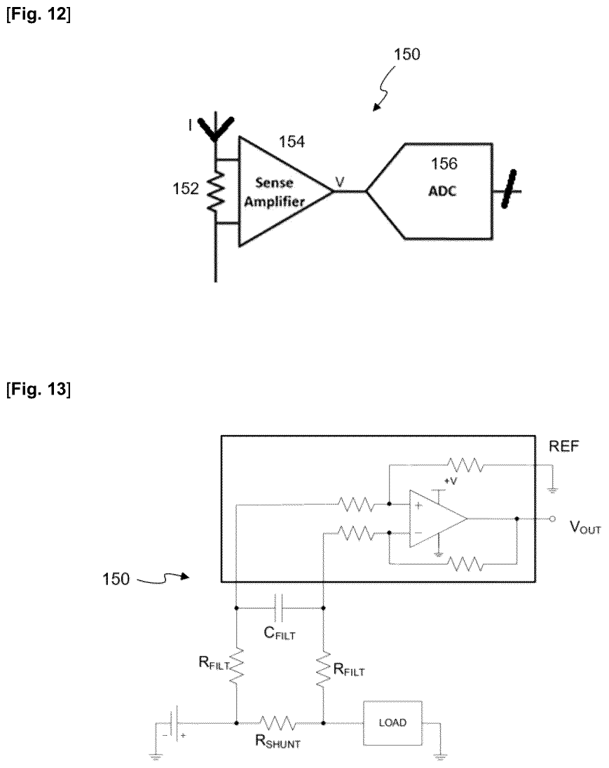

FIG. 12 schematically shows measurement means (or a measurement unit) 150 used by the control unit 140 (FIG. 10) to measure a current I in the system SY2, such as at least one of the abovementioned currents (current I2 at the pulser 101 output, supply current IHV+ and/or IHV− of the pulser 101, ground current IGND, etc.). As shown, these measurement means 150 comprise, for example, an operational amplifier 154, the two inputs of which are connected to the terminals of a resistor 152 through which a current I that is to be measured flows. This amplifier 154 generates an output voltage V that is representative of the current I flowing through the resistor 152. This voltage V can then be converted, for example, by an ADC (analogue-to-digital) converter with reference number 156 so that the control unit 140 can monitor the current I in question.

FIG. 13 schematically shows an exemplary embodiment of the measurement means 150 for measuring a current I in the system SY2.

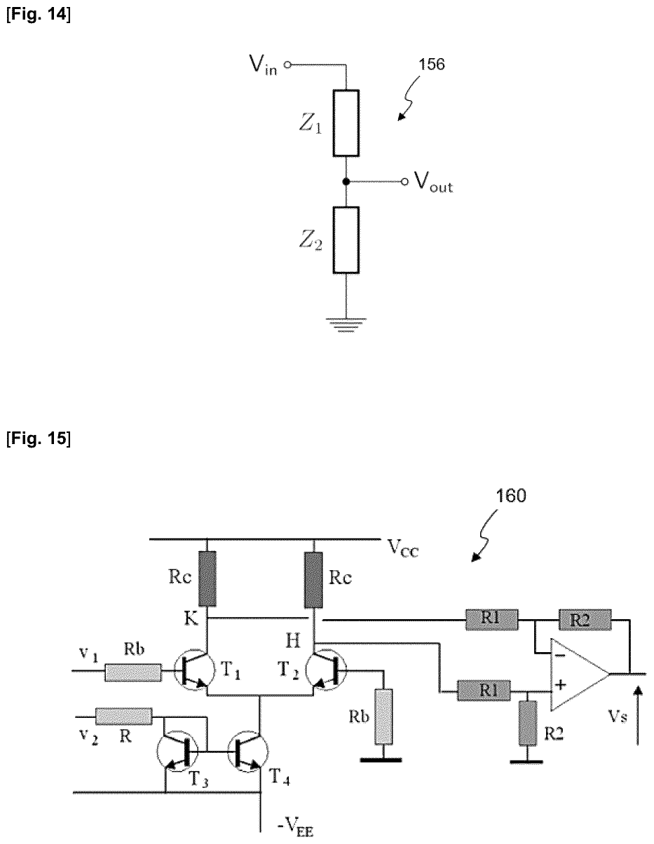

FIG. 14 schematically shows measurement means (or a measurement unit) 156 used by the control unit 140 (FIG. 10) to measure a voltage Vin in the system SY2, such as at least one of the abovementioned voltages (supply voltage HV− and/or HV+, voltage V2 at the pulser 101 output, voltage V3 at the terminals of the transmitting device 20, etc.). These measurement means 156 form a divider bridge from two impedances Z1 and Z2. The voltage Vin can be deduced from a measurement of the voltage Vout and the impedances Z1 and Z2.

FIG. 15 schematically shows measurement means (or a measurement unit) 160 used by the control unit 140 (FIG. 10) to measure a power, such as the power supplied to at least one of the supply terminals SHV+/SHV− or the power of the output signals SG1. As shown, these measurement means 160 comprise a circuit in which voltages V1 and V2 are received at the input, wherein V1 is a voltage acquired in the system SY2 and V2 is a voltage proportional to a current in the system SY2. This circuit allows the voltages V1 and V2 to be multiplied in order to obtain a voltage Vs at the output that is representative of a power (where R, Rb, Rc, R1 and R2 are resistors and where T1, T2, T3 and T4 are transistors).

As a person skilled in the art understands, the measurement means used by the control unit 140 can be adapted on a case-by-case basis, depending in particular of the type of operating parameters PR1 to be measured. By way of example, a hydrophone can be used to measure a sound pressure at the level of the transmitting device 20. A Hall effect sensor can be used to measure a magnetic field (or a variation of such a field) in the system SY2, for example at the level of the pulser 101 or the transmitting device 20. An electric field sensor (or probe) can also be used to measure an electric field in the system SY2.

According to one specific example, the control method implemented by the control system 100 or 200 comprises at least one of:
generating an alert if said at least one measured operating parameter PR1 (S4, FIG. 11) meets a predefined criterion; and
making, from said at least one measured operating parameter PR1, a prediction about the ageing of the transmitting device 20.

The above steps can be carried out, for example, by the control unit 140. In addition to controlling or adapting the modulation F1 of the output signals SG1 (FIGS. 10-11), it is thus possible to use the measurements of the operating parameters PR1 to generate alerts and/or make a prediction about the ageing of the transmitting device, which makes it possible to improve in particular the user experience and prevent misuse of the system.

As a person skilled in the art understands, all the embodiments and variants described above, some of which have been deliberately simplified to make them easier to explain, are merely non-limiting examples of the implementation of the present invention. In particular, a person skilled in the art will be able to envisage any adaptation or combination of the embodiments and variants described above, in order to meet a particular need.

The present invention is therefore not limited to the exemplary embodiments described above, but extends in particular to a control method that would include secondary steps without thereby going beyond the scope of the present invention. The same would apply to a control system for the implementation of such a method.

1. Method for controlling a transmitting device comprising a transducer for transmitting waves, said method comprising:
modulation of output signals emitted from a pulser in order to control the transducer, said modulation being performed by a controller according to at least one of:
a) control of a supply voltage of the pulser by a supply controller connected to a supply terminal of the pulser; and
control of return signals from the transducer by a return controller connected to a return terminal of the transducer.

2. Method according to claim 1, wherein during the control a), the supply controller on the supply terminal from a static voltage supplied by a voltage regulator.

3. Method according to claim 1, wherein the supply terminal of the pulser to which the supply controller is connected is one of: a positive supply terminal, a negative supply terminal and a ground terminal of the pulser.

4. Method according to claim 1, wherein the modulation is carried out by means of said supply controller, the modulation being configured to cause a variation over time of the supply voltage of the pulser according to at least two non-zero values.

5. Method according to claim 1, wherein the return terminal of the transducer is connected in series to a ground of the transmitting device.

6. Method according to claim 1, wherein the output signals are modulated at a modulation frequency Fm greater than or equal to a fraction 1/P of the pulse frequency Ft at which the transducer transmits waves in response to the output signals from the pulser, P being an integer equal to 10.

7. Method according to claim 1, wherein the transmitting device comprises a plurality of transducers each controlled by a respective pulser, signal modulation being performed independently per channel between each transducer and the respective pulser.

8. Method according to claim 1, wherein the method comprises:
measuring at least one operating parameter of a transmission chain via which the pulser sends the output signals to the transducer;
wherein the output signals are modulated as a function of said at least one measured operating parameter.

9. Method according to claim 8, said at least one operating parameter comprising at least one of:
a voltage at the pulser output;
the supply voltage of the pulser;
a ground voltage of the pulser;
a voltage at the terminals of the transmitting device;
a voltage received from the transducer;
a current at the pulser output;
a supply current of the pulser;
a ground current of the pulser;
a current flowing through the transmitting device;
a temperature of the pulser;
a temperature of the transmitting device;
an electric field emitted by the pulser;
an electric field emitted by the transmitting device;
a magnetic field emitted by the pulser;
a magnetic field emitted by the transmitting device;
a sound pressure emitted by the transmitting device; and
a sound pressure received by the transmitting device.

10. Method according to claim 8, wherein the method comprises:
comparing said at least one operating parameter with respectively one threshold value;
the modulation of the output signals being a function of a result of said comparison.

11. Method according to claim 10,
wherein the supply controller regulates the supply voltage to a first value during the control a) if said at least one operating parameter does not exceed a respective threshold value, and
wherein the control a) comprises adapting the supply voltage from the first value to a second value that is different from the first value if said at least one operating parameter exceeds said respective threshold value.

12. Method according to claim 8, wherein the output signals are modulated by slaving at least one of the controls a) and c) as a function of said at least one measured operating parameter.

13. Method according to claim 8, wherein said at least one operating parameter is measured during a calibration of the transmission chain, wherein the modulation is carried out after the calibration as a response to at least one command determined as a function of said at least one operating parameter.

14. Method according to claim 7, the method comprising:
measuring at least one operating parameter of transmission chains via which each transducer receives output signals from a respective pulser; and
upon detection that an operating parameter of a transmission chain meets a predetermined criterion, adapting the modulation of the output signals sent to the transducer of said transmission chain.

15. Method according to claim 1, wherein the output signals are modulated as a function of at least one of:
an imaging mode implemented by the pulser to control the transmitting device;
a type of transmitting device; and
user parameters of the transmitting device.

16. Method according to claim 1, wherein the transducer is a piezoelectric transducer.

17. Method according to claim 1, wherein said method is applied to medical ultrasound imaging.

18. Computer program comprising instructions for executing the steps of a method according to claim 1 when said program is run by a control system controlling a transducer device.

19. System for controlling a wave-transmitting device comprising a transducer for emitting waves, said system comprising:
a pulser; and
a controller configured to modulate output signals transmitted from the pulser to control the transducer, said controller comprising at least one of:
a) a supply controller, connected to a supply terminal of the pulser, configured to control a supply voltage of the pulser; and
c) a return controller, connected to a return terminal of the transducer, configured to control return signals from the transducer.
